(19) United States
(12) Patent Application Publication
(10) Pub. No.: US 2014/0158250 A1
(43) Pub. Date: Jun. 12, 2014

(54) PROCESS FOR FILLING GAS STORAGE CONTAINER

(71) Applicants: Neil Alexander Downie, Odiham (GB); Christopher John Mercer, Ash (GB)

(72) Inventors: Neil Alexander Downie, Odiham (GB); Christopher John Mercer, Ash (GB)

(73) Assignee: AIR PRODUCTS AND CHEMICALS, INC., Allentown, PA (US)

(21) Appl. No.: 13/993,793

(22) PCT Filed: Dec. 12, 2011
(86) PCT No.: PCT/EP2011/072455

§ 371 Date: Aug. 15, 2013

(30) Foreign Application Priority Data
Dec. 16, 2010 (EP) 10195491.5

(51) Int. Cl. F17C 5/02; A61K 33/00; C09K 3/30; A62D 1/00; C09K 5/04

(52) U.S. Cl. CPC F17C 5/02; A62D 1/0092; C09K 5/048; C09K 3/30; A61K 33/00
USPC 141/4; 252/8; 252/67; 516/7; 424/613

(57) ABSTRACT

A gas storage container, such as a gas cylinder, is filled with a gas mixture comprising a first gas and a second gas under pressure by feeding a liquid/solid mixture comprising liquefied first gas and solidified second gas into the gas storage container; closing the gas storage container to the passage of gas into or out from the container; and allowing said liquefied first gas and said solidified second gas to become gaseous within said closed gas storage container. Such a process is easier and more energy efficient as compared to direct compression processes, and is safer and results in less wastage as compared to direct liquid injection processes.

The present invention relates to a process for filling gas storage containers with a mixture of two or more gases. The gas storage containers are typically gas cylinders for storing and/or dispensing the gas mixtures under pressure, usually high pressure, e.g. at least 100 bar.

Mixtures of gases may be formed on site by mixing the individual gases in appropriate proportions. However, it may be more convenient to use a pre-mixed gas mixture stored in a container at high pressure.

Examples of gas mixtures in use every day include welding gases, such as argon/carbon dioxide/oxygen mixtures; “beer” gases, i.e. gases for use in pubs and bars to help dispense beer from pressurised metal kegs, such as nitrogen/carbon dioxide mixtures; anaesthetic gases, such as oxygen/nitrous oxide mixtures; and fire extinguishing gases, such as nitrogen/carbon dioxide mixtures.

Gas cylinders containing a gas mixture under high pressure, e.g. 100 bar or more, may be prepared by simply pumping a gas mixture into the cylinders using a gas compressor. Such a filling process tends to be used at sites where smaller numbers of cylinders are filled.

Examples of gas cylinders filled using a gas compressor include compressed air cylinders for diving which are prepared using a diving air compressor to compress air which is then fed to a cylinder.

U.S. Pat. No. 5,826,632 (published in October 1998) discloses a method for filling gas storage vessels with a gas mixture. The method involves providing a flow of a uniformly blended gas mixture under pressure, monitoring the flow rate and composition of the mixture, and adjusting the flow rate and/or composition as appropriate to maintain the required proportions of the gases in the gas mixture. The gas mixture is then fed to one or more gas cylinders. U.S. Pat. No. 5,826,632 exemplifies preparing gas cylinders containing 90% argon/10% carbon dioxide at 182 bar.

Gas cylinders containing a gas mixture under high pressure may also be prepared by feeding sequentially each component of the gas mixture into the cylinder. The method involves measuring either the increase in partial pressure in the cylinder (manometric method), or the increase in mass of the cylinder (gravimetric method) during the addition of each component.

Manometric methods can be inaccurate, particularly for non-ideal gases, and usually involve the use of different types of pressure gauge for low and high pressures. Changing pressure gauges is labour intensive and extends the time taken to fill a cylinder. In addition, such pressure gauges are typically expensive.

Gravimetric methods are typically more accurate that manometric methods. However, they still involve the use of expensive equipment and can be quite complicated.

U.S. Pat. No. 5,427,160 (published in June 1995) discloses a method of filling a gas storage container with a combustible mixture of gases. The method preferably involves conducting a flammable gas under pressure from a first intermediate container thereof to a gas storage vessel, and then conducting an oxidizer gas under pressure from a second intermediate container thereof to the gas storage vessel. The flow of gas to the storage vessel is controlled by suitable valves and pressure transducers. U.S. Pat. No. 5,427,160 exemplifies preparing gas storage containers intended for use in a vehicle air bag system, containing a mixture of air (with oxygen in the air acting as the oxidizer gas) and hydrogen as the flammable gas at a pressure of 2,500 psi (˜172 bar).

One drawback of methods involving the sequential addition of components of the gas mixture is that the gases in the cylinder may stratify until the gas mixture reaches equilibrium. Such stratification may be overcome by introducing the lighter component(s) to the bottom of the cylinder by means of a mixing tube, or by rolling the filled cylinder. Another option is to charge the desired quantities of each gas into the cylinder in the order of their increasing densities. U.S. Pat. No. 5,353,848 (published in October 1994) discloses such a method.

A significant drawback of direct compression methods is that each cylinder must be filled slowly, e.g. less than 1 bar/s, to control and/or minimise the heating of the cylinder by adiabatic compression of the gas. Filling a cylinder with a gas mixture can take 1-2 h and is, therefore, one of the rate-limiting steps in preparing high pressure gas cylinders. In addition, a significant amount of energy is required to compress the gas to sufficient pressure to fill the cylinder. Further, the capital and operating costs of high pressure compressors are typically high.

A further drawback of direct compression methods is that heat of compression may have a significant adverse effect on the metering precision with which the flow of gas into the cylinder is monitored. Obviously, this effect is not desirable since the composition of the gas mixture is typically critical.

US 2008/0202629 (published in August 2008) discloses a two-step method for preparing a gas container containing a gas mixture under high pressure, involving supplying a liquefied or solidified first gas into a gas container while the gas container is being cooled, and then introducing a second gas into the gas container before closing the gas container. After closure, the container may be warmed up to ambient temperature whereupon the liquefied or solidified first gas becomes gaseous, thereby increasing the pressure inside the container. The pressure at 15° C. in the container may be from 250 bar to 1300 bar. The method is particularly applicable for preparing high pressure gas containers for air bag systems involving gases such as argon, oxygen, nitrogen, hydrogen, helium, dinitrogen monoxide (N₂O) as pure gases or mixtures, and it is disclosed that advantageously the first gas may be argon, and the second gas may be helium. US 2008/02026289 discloses that the method allows for tighter metering control of the components of the gas mixture.

Certain cryogenic slurries comprising solid CO₂ and a cryogenic liquid are known in the art. For example, U.S. Pat. No. 3,393,152 (published in July 1968) discloses a refrigerant composition comprising solid carbon dioxide particles suspended in a cryogenic liquid having a boiling temperature below about −300° F. (˜184° C.). The preferred cryogenic liquid is liquid nitrogen although it is disclosed that liquid air or liquid argon may be used. The proportion of solid carbon dioxide in the composition may be from 5 wt % to 95 wt % although, where higher refrigeration capacity is required, a proportion of above 40 wt % is preferred. U.S. Pat. No. 3,393,152 exemplifies forming the composition either by passing compressed carbon dioxide gas through liquid nitrogen in a pressure tank, or by expanding liquid carbon dioxide to produce carbon dioxide snow which then falls directly on to liquid nitrogen within which it becomes suspended. It is disclosed that the composition is useful as a refrigerant and as a source of inert gas. The composition may be also used as a cooling medium and as such may be used in diverse fields such as welding and blow molding.

U.S. Pat. No. 5,368,105 (published in November 1994) discloses a cryogenic slurry for use as a fire extinguishant. The slurry comprises a mixture of solid carbon dioxide particles suspended in liquid nitrogen in a ratio of about 1:1 by weight.

WO 00/36351 (published in June 2000) discloses a cryogenic slurry containing solid carbon dioxide particles (e.g. 10-50 wt %) suspended in a mixture of liquid nitrogen (or liquid air; e.g. 50-90 wt %) and ethanol (e.g. 20-60 wt %). It is disclosed that the composition may have a Vaseline-like or cream-like consistency, and may be used to treat warts, freeze-seal pipelines and cool laboratory samples. WO 00/36351 also speculates that the mixture may be used to replace dry ice in a number of areas, and suggests that the good weight/cool properties of the mixture means that it can be in the transportation/storage of frozen/refrigerated products such as foods.

It is an objective of preferred embodiments of the present invention to provide a new method of filling a gas storage container with a gas mixture under pressure, preferably without one of more of the drawbacks of the prior art.

According to a first aspect of the present invention, there is provided a process for filling a gas storage container with a gaseous mixture of at least a first gas and a second gas under pressure, said process comprising:

charging a gas storage container with a liquid/solid mixture comprising liquefied first gas and solidified second gas;

closing said gas storage container to the passage of gas into or out from the container; and

allowing said liquefied first gas and said solidified second gas to become gaseous within said closed gas storage container.

The Inventors have observed that liquid argon/solid carbon dioxide slurries do not boil so readily as liquid argon itself when fed to a gas cylinder. Suppression of boiling during fill means that higher pressure fills can be achieved, or lower pressures are needed when injecting cylinders with slurry versus pure cryogenic liquid. In addition, loss of cryogenic fluid during fill is reduced.

Without wishing to be bound by any particular theory, the Inventors believe that this observation arises because of the specific heat capacity of solid carbon dioxide which provides additional capacity for refrigeration. The Inventors fully expect that similar boiling suppression effects should be observed with other cryogenic slurries involving different mixtures of liquefied gas(es) and solidified gas(es), e.g. liquid nitrogen/carbon dioxide.

The Inventors have also observed that, where solid carbon dioxide is present in the liquid/solid mixture, the solid carbon dioxide appears to suppress immediate boiling of the liquefied first gas, and since the mixture has a higher viscosity that the liquefied first gas alone, there is less “splashing” of the liquefied first gas during fill.

The term “under pressure” is intended to mean that the gas mixture is at a pressure that is above atmospheric pressure, e.g. at least 40 bar. The container is typically suitable for storing and/or dispensing gas up to a pressure of about 500 bar. Usually, the container is suitable for storing and/or dispensing gas at a pressure of at least 100 bar, e.g. at least 200 bar, or at least 300 bar.

The liquid/solid mixture is typically stable for at least 10 mins, preferably at least 30 mins, and more preferably up to 1 hour, at ambient pressure, e.g. from about 1 to about 2 bar. The term “stable” in this context means that the mixture may be handled at ambient pressure without significant loss of one of more of the components.

The liquid/solid mixture is typically fluid enabling the mixture to be poured, pumped/piped along a conduit, and valved. Depending on the relative proportions of liquefied gas(es) and solidified gas(es), the consistency and appearance of the mixture may range from a thick, creamy substance (not unlike whipped cream or white petrolatum) to a thin, milky substance. The range of viscosity of the mixture is typically from about 1 cPs (for thin, milky mixtures) to about 10,000 cPs (for thick, creamy mixtures). The viscosity may be from about 1,000 to about 10,000 cPs. Preferably, the mixture is composed of finely divided solid particles suspended in a liquid phase. The liquid/solid mixture may be described as a cryogenic slurry or slush.

The Inventors have observed that, when a liquid argon/solid carbon dioxide mixture is allowed to warm to ambient temperature, the liquid argon evaporates first to leave a substantial amount of the solid carbon dioxide behind which then gradually sublimes. A uniformly blended argon/carbon dioxide mixture is formed by diffusion of the gases within the container. The Inventors expect that other liquid/solid mixtures containing solid carbon dioxide will behave in a similar manner.

The relative proportions of the liquid and solid components in the mixture are dictated by the desired gas mixture and by the desire for the mixture to have fluid characteristics. In preferred embodiments, there is from about 40 wt % to about 99 wt % liquid component(s) and from about 1 wt % to about 60 wt % solid component(s).

The identities of the first and second gases will be dictated by the gas mixture filling the container. Examples of suitable gas mixtures for use with the present invention include welding gases; “beer” gases; anaesthetic gases; and fire extinguishing gases.

Suitable welding gases include nitrogen/carbon dioxide mixtures (e.g. from about 80 wt % to about 95 wt % nitrogen and from about 5 wt % to about 20 wt % carbon dioxide), and argon/carbon dioxide mixtures (e.g. from about 80 wt % to about 95 wt % argon and from about 5 wt % to about 20 wt % carbon dioxide). Oxygen may replace some of the nitrogen or argon gas in such welding gas mixtures. Thus, the welding gases may contain from 0 wt % to about 5 wt % oxygen.

A particularly suitable welding gas contains from about 80 wt % to about 90 wt % argon, from 0 wt % to about 5 wt % oxygen, and from about 5 wt % to about 20 wt % carbon dioxide. An example of a suitable welding gas contains about 2.5 wt % oxygen, from about 7 wt % to about 20 wt % carbon dioxide with the balance (from about 77.5 wt % to about 90.5 wt %) being argon.

Suitable “beer” gases include nitrogen/carbon dioxide mixtures (e.g. from about 40 wt % to about 70 wt % nitrogen and from about 30 wt % to about 60 wt % carbon dioxide).

Suitable anaesthetic gases include oxygen/nitrous oxide mixtures (e.g. from about 65 wt % to about 75 wt % oxygen and from about 25 wt % to about 35 wt % nitrous oxide).

Suitable fire extinguishing gases include nitrogen/carbon dioxide mixtures (e.g. in a weight ratio of 1:1).

The first gas may therefore be selected from the group consisting of nitrogen; argon; and oxygen. Other suitable gases include helium; neon; xenon; krypton; and methane.

The second gas is typically stable in solid form at ambient pressure. The term “stable” in this context means that the solid form of the second gas does not become gaseous (either by sublimation, or by melting and evaporation) unduly rapidly at ambient pressure so that the solid form may be handled easily under these conditions. The second gas is typically selected from the group consisting of carbon dioxide and nitrous oxide.

The liquid/solid mixture may be a binary mixture of a liquefied gas and a solidified gas. However, the liquid/solid mixture may be a mixture of more than one liquefied gas and one solidified gas, or a mixture of one liquefied gas and more than one solidified gas. In some preferred embodiments, the liquid/solid mixture comprises a liquefied third gas. The liquefied third gas may be immiscible with the liquefied first gas but, in preferred embodiments, the liquefied first and third gases are miscible with each other.

In preferred embodiments in which the gas storage container is filled with a welding gas, the liquefied first gas is liquid argon, and the solidified second gas is solid carbon dioxide. In such embodiments, the liquid/solid mixture may also comprise liquid oxygen which is miscible with liquid argon. Thus, the liquid/solid mixture may comprise from about 80 to about 90 wt % liquid argon; from 0 to about 5 wt % liquid oxygen; and from about 5 to about 20 wt % solid carbon dioxide.

The present invention may be applied to any type of container for storing and/or dispensing gas under pressure, such as gas tanks or other gas storage vessels. The gas storage container typically comprises an outer vessel defining an interior space for holding a gas mixture under pressure, said outer vessel comprising an opening for receiving a fluid flow control unit; and a fluid flow control unit mounted within said opening for controlling fluid flow into and out of the outer vessel.

The present invention has particular application to gas cylinders, e.g. high pressure gas cylinders made from, for example, steel or aluminium. In some preferred embodiments, the container is a single gas cylinder. In other preferred embodiments, the container is a central “primary” cylinder in parallel gas flow communication with a plurality of “secondary” cylinders in a multi-cylinder pack. In such embodiments, the outer vessel of the central cylinder is usually made from aluminium, and the outer vessel of each secondary cylinder is usually made from steel.

The gas storage container may be a cylinder having an inner surface lined with heat insulation material. A suitable example of such a cylinder is described in GB 2,277,370, the disclosure of which is incorporated herein by reference. However, the gas storage container is preferably unlined.

The gas storage container may also comprise at least one inner vessel provided within said interior space, said inner vessel(s) defining a part of said interior space for holding the liquid/solid mixture in spaced relationship with said outer vessel and being in fluid flow communication with a remaining part of said interior space. Such an arrangement prevents embrittlement of the outer vessel.

In these embodiments, the cryogenic fluid is fed to the inner vessel(s) inside the container. The container is then sealed and the cryogenic fluid is then allowed to become gaseous thereby filling the container, and any secondary containers associated therewith, with gas under pressure. The inner vessel(s) not only isolate the cryogenic fluid from the outer wall of the container (thereby preventing embrittlement of the container), but since they tend to be thin walled also reduce the rate of boiling and provide more uniform boil off.

The or each inner vessel is preferably “loose-fitting”, i.e. not fixedly mounted within the container.

The or each inner vessel is preferably “thin-walled” since the inner vessel(s) is exposed only to isostatic pressure. The or each inner vessel usually has a base and enclosing wall(s) that are sufficiently thick such that the inner vessel is able to support itself when containing cryogenic fluid. The thickness of the base and enclosing wall(s) depend on the material from which the inner vessel is made but, typically, the base and wall(s) of the inner vessel(s) have a thickness from about 0.1 mm to about 10 mm, preferably from about 0.25 mm to about 5 mm. For example, where an inner vessel is made from a metal, e.g. steel, aluminium or nickel, the thickness of the base and wall(s) is typically no more than about 2 mm, e.g. from about 1 mm to about 2 mm. In addition, where the inner vessel is made from a polymeric material, e.g. silicone or polyester film, the thickness of the base and the wall(s) is typically a little more, e.g. less than about 5 mm, e.g. from about 1.5 mm to about 4 mm.

The or each inner vessel is preferably in the form of an “open-topped” or “open-ended” can, i.e. a vessel having a base and an enclosing wall, typically (although not necessarily) circular, provided substantially perpendicular to the base. The mouth of such an inner vessel is the open end. In some embodiments, the open end of said can is in the form of an inverted cone.

The gas storage container preferably comprises at least one support for supporting the inner vessel(s) in said spaced relationship with respect to said outer vessel. Any suitable support may be used such as spacer arms and/or legs for the inner vessel(s), or a support base on which the inner vessel(s) sits. The support(s) may be (although are not necessarily) fixed to the inner vessel(s). The or each support is usually made from a cryogenic resistant material, and typically has a low heat transfer coefficient. Suitable materials include plastics and polymers, but packing material may also be used.

The container may comprise a plurality of inner vessels. For example, each inner vessel may be a long thin-walled pipe having a closed bottom end and an open top end forming the mouth. The diameter of the pipe may be more than the diameter of the opening of the outer vessel (in which case, the pipes would be introduced into the outer vessel prior to enclosure) or less than that diameter of the opening in the outer vessel (in which case, each pipe could be inserted into the outer vessel via that opening).

In preferred embodiments, the container comprises a single inner vessel. In such embodiments, the mouth of the inner vessel preferably has a diameter that is greater than that of said opening. The diameter of the mouth of the inner vessel may be at least 100% greater, preferably at least 200% greater, e.g. at least 400% greater, than that of the opening. The diameter of the mouth of the inner vessel may be up to about 99% of the internal diameter of the outer vessel.

The or each inner vessel is usually self-supporting, even when charged with cryogenic fluid. The inner vessel(s) may be rigid, i.e. self-supporting and possibly resistant to deformation. Alternatively, the or at least one of the inner vessels may be deformable. In such embodiments, the or each inner vessel may be deformed, e.g. by rolling, folding or crushing, and then inserted into the container through the opening in the outer vessel. The or each inner vessel may then be unfurled inside the container using gas pressure or hydraulic pressure. Alternatively, in embodiments where the or each inner vessel is resilient, the inner vessel resumes its original shape unaided inside the container. In this connection, either the inner vessel is made from a resilient material or the inner vessel comprises an inherently resilient, or “spring-loaded”, frame supporting a deformable sheet material forming the base and walls of the vessel.

Since it is to be charged with cryogenic fluid, the or each inner vessel is typically made from a material that is resistant to embrittlement at the cryogenic temperatures to which it will be exposed. Suitable materials include specific metals, e.g. aluminium; nickel; and steel, for example, stainless steel; and polymeric materials, e.g. silicones such as catalytically set silicone and polydimethylsiloxanes; polyesters such as polyethylene terephthalate (PET or Mylar™); polyethylenes such as polytetrafluoroethylene (PTFE); and perfluorinated elastomers (PFE).

The inner vessel may comprise at least one aperture, in addition to the mouth, for providing additional gas flow communication between the part of the interior space defined by the inner vessel and the remaining part of the interior space defined by the outer vessel. Such aperture(s) would typically be provided in the wall of the inner vessel, above the maximum level of cryogenic fluid to be charged to the vessel. However, in preferred embodiments, the mouth is preferably the sole opening in the or each inner vessel.

The term “spaced relationship” is intended to mean spaced apart from or having a gap therebetween. Thus, in the present invention, there the outer vessel is spaced apart from the inner vessel(s) such that the cryogenic fluid charged to the inner vessel(s) is isolated from the outer vessel by a gap provided therebetween. The gap is usually more than 1 mm, and preferably more than 5 mm.

The term “open” is intended to mean at least not entirely closed. Thus, in the present invention, the mouth is at least not entirely closed and, preferably entirely open, to the remaining part of the interior space. In preferred embodiments, the mouth is free of direct attachment to any part of the container, particularly the fluid flow control unit.

The mouth of the or each inner vessel is preferably in spaced relationship with respect to the fluid flow control unit.

The interior space typically has a top half and a bottom half. The extent to which the inner vessel extends into the bottom half or top half of the interior space depends on the amount of cryogenic fluid to be charged to the inner vessel. The or each inner vessel may extend from the bottom half into the top half of the interior space. For example, in embodiments in which the container is the central primary cylinder in a multi-cylinder pack, the inner vessel may extend essentially from near the bottom of the interior space to the top, or up to 90% of the length of the interior space. However, in embodiments in which the container is an individual gas cylinder, the inner vessel is preferably provided entirely within the bottom half, or even bottom third, of the interior space.

Certain preferred containers for storing and/or dispensing gas under pressure are disclosed in co-pending European patent application No. (to be advised) and identified under APCI Docket No. 07492 EPC, the disclosure of which is incorporated herein by reference.

The Inventors have discovered that an inner vessel in the form of an open-topped can is superior to an inner vessel in the form of a bag sealed at the mouth since the bag inhibits diffusion of the second gas necessary to form a uniformly blended gaseous mixture. In addition, the Inventors have observed that use of the internal can in the base of the container avoids the fierce convection encountered if the mixture is fed to an internal bag connected to the fluid flow control unit. Further, the Inventors have observed that an internal can is more robust that an internal bag.

The gas storage container, or the inner vessel(s) provided therein, may be charged with the liquid/solid mixture using a nozzle inserted into a passageway through the fluid flow control unit. The nozzle typically comprises a first conduit arrangement through which the cryogenic fluid is fed, and a second conduit arrangement through which displaced air and/or gaseous cryogenic fluid is vented from the container when charging the fluid to the container. The first conduit arrangement may be within and preferably coaxial with the second conduit arrangement. In embodiments where an inner vessel is spaced apart from the fluid flow control unit, the nozzle typically extends through the fluid flow control unit to below the level of the mouth of the inner vessel. In this way, spray from the end of the nozzle is caught by the walls of the inner vessel.

The passageway may be opened and closed manually using a pressure cap or alike although in preferred embodiments, the passageway has a valve located at the end of the passageway inside the container that is biased in the closed position by a spring.

The process may comprise opening the passageway by removing the pressure cap, and then inserting a nozzle into the open passageway and feeding the cryogenic fluid into the container. Alternatively, the process may comprise opening the passageway by inserting the nozzle with the end of the nozzle pushing open the valve against the spring.

Suitable nozzle arrangements are disclosed in co-pending European patent application No. 10 195 461.8 filed on 16 Dec. 2010, the disclosure of which is incorporated herein by reference.

The liquid/solid mixture may be produced by contacting the second gas with the liquefied first gas. The second gas may be in gaseous form although is typically in the form of liquefied or solidified particles.

The liquid/solid mixture may be formed by passing the second gas under pressure through liquefied first gas in an insulated tank. The liquefied first gas cools and solidifies the second gas in the form of finely divided solid particles which then disperse within the liquefied first gas. A suitable example of such a process is described in U.S. Pat. No. 3,393,152, the disclosure of which is incorporated herein by reference.

The liquid/solid mixture may also be formed by rapidly expanding a stream of pressurised second gas in either gaseous or liquid form and mixing the expanded stream with a spray of liquefied first gas. Suitable examples of such a process are described in U.S. Pat. No. 5,368,105 and WO 00/36351, the disclosures of which are incorporated herein by reference. The Inventors note that a nozzle for liquid carbon dioxide may be heated to avoid blockage with solid carbon dioxide.

The Inventors produced a liquid argon/solid carbon dioxide mixture by discharging carbon dioxide from a cylinder containing pressurised carbon dioxide, over liquid argon. The carbon dioxide liquefies/solidifies when discharged to form fine droplets/particles which then fall on to the surface and are mixed with the liquid argon. The Inventors have observed that mixtures made this way should be “milky” if they are to be sufficiently stable to enable charging to a gas cylinder.

The liquid/solid mixture may be produced in batches in tanks, or in a continuous in-line process. The mixture may be metered gravimetrically, or using a flowmeter such as a coriolis flowmeter.

The amount of the liquid/solid mixture fed, or charged, to the gas storage container is calculated to provide the desired pressure of gas mixture in the container once the mixture becomes gaseous.

Where a gas storage container is to be filled with gas under pressure, the quantity of cryogenic liquid to the charged to the inner vessel(s) can be calculated using the ideal gas equation, viz:

PV=nRT

where P is the desired pressure of the gas in the container; V is the volume of the container; n is the number of moles of gas; R is the gas constant; and T is the absolute temperature.

Once a particular container is selected, V and the maximum P are known, as is R and the ambient temperature. The value of n may then be calculated thus:

n=PV/RT

The number of moles, n, of gas is then converted into mass, M, of gas in grams (g) by multiplying by the molecular weight, A:

M=nA

For real gases at pressure above say 50 bar, there are corrections to be added to this basic formula which depend upon the attractive and repulsive forces between molecules, and the finite and different size of molecules. These corrections can be taken account of by including a factor Z, the “compressability” of the gas, in the equation:

PV=nRTZ

Tabulations exist for many gases over a wide range of pressures and temperatures, and complex approximate formulae exist for some gases.

The calculation may be adapted as appropriate to determine the amount of a liquid/solid mixture comprising a liquefied first gas and a solidified second gas, that would be required to fill a gas storage container with a gas mixture under pressure.

In a batch process, the pre-determined amount may be measured out (e.g. gravimetrically or volumetrically) and then charged to the container using for example a funnel or a siphon. Alternatively, in a continuous process using a filling line, a flow of the liquid/solid mixture to a first container may be metered (e.g. using a flowmeter, or by a gravimetric or volumetric method) and, once the pre-determined amount has been charged to the first container, the flow may be interrupted to allow the first container to be closed and removed from the line, and a second container to be moved into position ready to be charged with the liquid/solid mixture.

Charging the cryogenic liquid/solid mixture to the inner vessel(s) of a single container usually takes no more than 1 min and may take a little as 10 to 20 s.

The gas storage container is typically allowed to stand at ambient temperature for at least sufficient time to permit the mixture to become gaseous and for the gases to diffuse to provide a uniformly blended gas mixture. In this connection, the gas storage container may be allowed to stand from about 12 h up to a week to ensure complete diffusion. Diffusion may be enhanced or promoted by lying the container, e.g. cylinders, horizontally, or by moving the container, e.g. by rolling.

According to a second aspect of the present invention, there is provided a liquid/solid mixture comprising liquid argon, liquid oxygen, and solid carbon dioxide.

The liquid/solid mixture preferably comprises from about 80 to about 90 wt % liquid argon; more than 0 wt %, e.g. from about 0.1 wt %, to about 5 wt % liquid oxygen; and from about 5 to 20 wt % solid carbon dioxide. A preferred liquid/solid mixture consists essentially of liquid argon, liquid oxygen and solid carbon dioxide in these proportions.

The following is a description, by way of example only and with reference to the accompanying drawing, of a presently preferred embodiment of the present invention. Regarding the drawings:

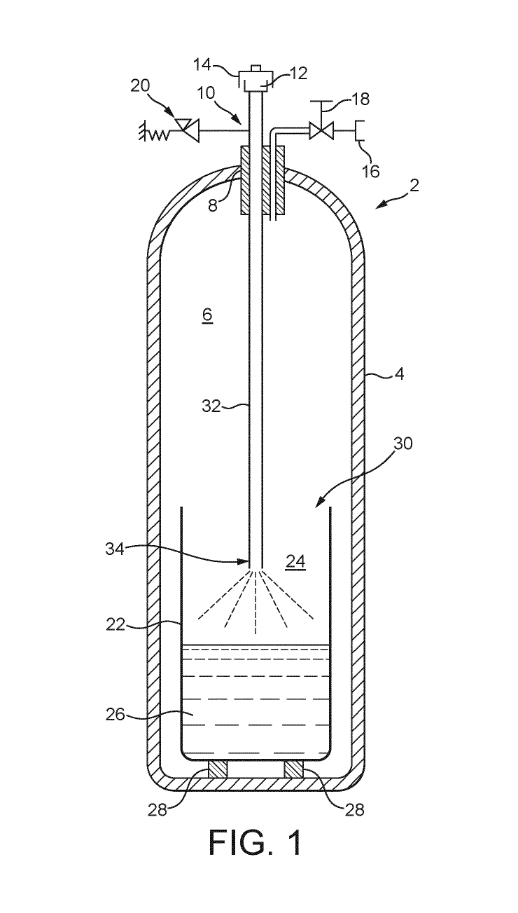

FIG. 1 is a schematic cross-sectional representation of one embodiment of a gas storage container according to the present invention; and

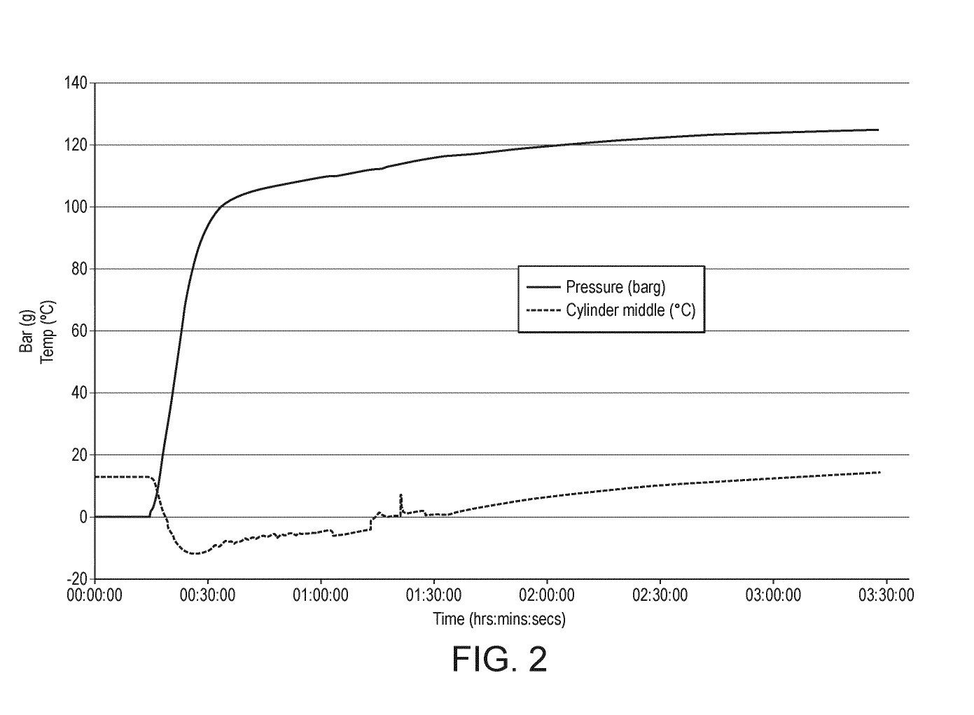

FIG. 2 is a graph depicting (i) an accelerating pressure curve over time for a gas cylinder having an interior bag charged with a cryogenic slurry formed from liquid argon and solid carbon dioxide, and (ii) temperature variations over time at different points on the cylinder.

Regarding FIG. 1, a gas cylinder 2 has an outer vessel 4 defining an interior space 6 for holding gas under pressure. The outer vessel 4 is made from steel and has an opening 8 for receiving a fluid flow control unit 10 for controlling fluid flow into and out of the cylinder 2. The fluid flow control unit 10 has a fill inlet 12 suitable for filling a liquid/solid mixture of a liquefied first gas and a solidified second gas into the cylinder, with a pressure cap 14, and a customer outlet 16 having a control valve 18. The fluid flow control unit 10 also has a pressure relief valve 20.

An inner vessel 22 made from aluminium is provided entirely within the bottom half of the interior space 6. The inner vessel 22 defines a part 24 of the interior space for holding cryogenic fluid 26 in spaced relationship with respect to the outer vessel. A support 28 provides the spaced relationship between the inner vessel 22 and the outer vessel 4. The inner vessel 22 has a mouth 30 for receiving the liquid/solid mixture from the fluid flow control unit 10 via a conduit 32, or dip tube, made from aluminium. The end 34 of the conduit 32 extends below the mouth 30 of the inner vessel 22, thereby ensuring that spray from the conduit 32 is caught by the inner vessel 22. The end 34 of the conduit 32 does not usually extend so far below the mouth 30 of the inner vessel 22 such that it would be below the surface of the liquid/solid mixture 26 after the inner vessel 22 has been charged with the mixture.

The mouth 30 is open to the remaining part of the interior space 6 and thereby provides fluid flow communication between the inner vessel 22 and the remaining part of the interior space 6.

The cylinder 2 is filled by removing the pressure cap 14 and feeding liquid/solid mixture down the conduit 32 into the inner vessel 22. The control valve 18 on the customer outlet 16 may be open to allow displaced gas to escape from the cylinder 2.

The amount, e.g. volume or mass, of the liquid/solid mixture to be fed to the cylinder 2 is pre-determined based on the target pressure of the gas in the cylinder (and, hence, the volume of the cylinder, the densities of the liquefied first gas and solidified second gas, and the gas mixture), and feed to the cylinder is metered to ensure that the correct amount of cryogenic fluid is added. Once the required amount of the liquid/solid mixture has been added to the cylinder 2, the inlet 12 is closed off with the pressure cap 14, and the control valve 18 in the customer outlet 16 is closed. The mixture is then allowed to become gaseous by evaporation and where appropriate by sublimation, thereby filling the cylinder 2 with gas to the desired pressure.

### Example

A 23.5 L steel gas cylinder having a large (40 mm) neck was equipped with a fluid flow control unit having a cryogenic fluid filling aperture and tube, a customer valve and a safety relief valve. A Mylar™ bag was connected to the liquid filling tube and provided inside the cylinder. The resultant cylinder and internals were similar to the type described in U.S. Pat. No. 3,645,291.

A slurry of 97 wt % liquid argon/7 wt % solid carbon dioxide was prepared by spraying liquid carbon dioxide from a nozzle on to the surface of a vented tank of liquid argon. After sufficient carbon dioxide had been added, the resultant slurry was checked for free-flowing characteristic and colour. An opaque white watery liquid was achieved.

The system was pre-cooled with LIN before filling. After pre-cooling, about 4.2 litres (6 litres total with a loss of 1.8 litres due to blow back and spitting, etc.) of the mixture was poured through the central tube in a coaxial nozzle into the fill tube and the bag. The customer valve was open when the mixture was poured in, and then both the customer valve and the liquid filling aperture closed after the mixture had been poured in. The pressure and temperature of the cylinder were then logged over time. Carbon dioxide content was measured every few hours over several days until it returned to an equilibrium value of 7%.

The graph in FIG. 2 depicts how the observed pressure inside the cylinder increases over time as the LAr/CO₂ slurry becomes gaseous. The pressure inside the cylinder increases rapidly over the first 30 seconds due primarily to evaporation of the LAr from the slurry. After about 30 seconds, substantially all of the LAr has evaporated. The pressure continues to increase (albeit at a lower rate) due to sublimation of the solid CO₂ left over from the slurry after the liquid argon has evaporated.

The graph in FIG. 2 also indicates that the temperature at the coldest point of the cylinder (the middle) does not drop below −20° C. at any point during the filling process. These results indicate that the outer vessel of the cylinder can be made from materials such as steel which tend to be less resistant to cryogenic temperatures.

The Inventors expect that the loss of mixture due to blow back and spitting, etc. would be significantly reduced if the mixture is charged to an internal can in the base of the cylinder.

Advantages of preferred embodiments of the present invention include:
Easier and more rapid filling of a gas storage container with a gas mixture when compared to direct compression processes;
More energy efficient filling of gas storage containers when compared to direct compression processes;
More reliable and safer filling of gas storage vessels when compared to direct liquid injection processes; and
Less wastage of liquefied gases during filling of gas storage containers.

It will be appreciated that the invention is not restricted to the details described above with reference to the preferred embodiments but that numerous modifications and variations can be made without departing form the spirit or scope of the invention as defined in the following claims.

1. A process for filling a gas storage container with a gaseous mixture of at least a first gas and a second gas under pressure, said process comprising:
charging a gas storage container with a liquid/solid mixture comprising liquefied first gas and solidified second gas;
closing said gas storage container to the passage of gas into or out from the container; and
allowing said liquefied first gas and said solidified second gas to become gaseous within said closed gas storage container.

2. A process as claimed in claim 1, wherein the first gas is selected from the group consisting of nitrogen (N2); argon (Ar); oxygen (02); helium; neon; krypton; methane; and mixtures thereof.

3. A process as claimed in claim 1, wherein the second gas is selected from the group consisting of carbon dioxide (C02) and nitrous oxide (N20).

4. A process as claimed in claim 1, wherein the mixture comprises from about 40 wt % to about 99 wt % liquid component(s) and from about 1 wt % to about 60 wt % solid component(s).

5. A process as claimed in claim 1, wherein said liquid/solid mixture comprises a liquefied third gas.

6. A process as claimed in claim 5, wherein said liquefied third gas is miscible with the liquefied first gas.

7. A process as claimed in claim 1, wherein the gaseous mixture is a welding gas.

8. A process as claimed in claim 1, wherein said liquefied first gas is liquid argon, and said solidified second gas is solid carbon dioxide.

9. A process as claimed in claim 8, wherein said liquid/solid mixture comprises liquid oxygen.

10. A process as claimed in claim 9, wherein said liquid/solid mixture comprises:
from about 80 to about 90 wt % liquid argon;
from 0 to about 5 wt % liquid oxygen; and
from about 5 to about 20 wt % solid carbon dioxide.

11. A liquid/solid mixture comprising liquid argon, liquid oxygen, and solid carbon dioxide.

12. A liquid/solid mixture as claimed in claim 11, comprising:
from about 80 to about 90 wt % liquid argon;
up to about 5 wt % liquid oxygen; and
from about 5 to 20 wt % solid carbon dioxide.

13. A process for filling a gas storage container with a gaseous mixture of at least a first gas and a second gas under pressure, said process comprising:
charging a gas storage container with a liquid/solid mixture comprising liquefied first gas and solidified second gas, wherein the first gas is selected from the group consisting of nitrogen (N2); argon (Ar); oxygen (02); helium; neon; krypton; methane; and mixtures thereof, wherein the second gas is selected from the group consisting of carbon dioxide (C02) and nitrous oxide (N20);
closing said gas storage container to the passage of gas into or out from the container; and
allowing said liquefied first gas and said solidified second gas to become gaseous within said closed gas storage container.

14. A process as claimed in claim 13, wherein the liquid/solid mixture comprises from about 40 wt % to about 99 wt % liquid component(s) and from about 1 wt % to about 60 wt % solid component(s).

15. A process as claimed in claim 13, wherein said liquid/solid mixture comprises a liquefied third gas.

16. A process as claimed in claim 15, wherein said liquefied third gas is miscible with the liquefied first gas.

17. A process as claimed in claim 13, wherein the gaseous mixture is a welding gas.

18. A process as claimed in claim 13, wherein said liquefied first gas is liquid argon, and said solidified second gas is solid carbon dioxide.

19. A process as claimed in claim 18, wherein said liquid/solid mixture comprises liquid oxygen.

20. A process as claimed in claim 19, wherein said liquid/solid mixture comprises:
from about 80 to about 90 wt % liquid argon;
from 0 to about 5 wt % liquid oxygen; and
from about 5 to about 20 wt % solid carbon dioxide.
